(19) United States
(12) Patent Application Publication
(10) Pub. No.: US 2024/0270924 A1
(43) Pub. Date: Aug. 15, 2024

(54) Residential Plastic Recycling Unit

(71) Applicants: Laura Kolinsky, Roslindale, MA (US); Markian Kolinsky, Roslindale, MA (US)

(72) Inventors: Laura Kolinsky, Roslindale, MA (US); Markian Kolinsky, Roslindale, MA (US)

(21) Appl. No.: 18/462,547

(22) Filed: Sep. 7, 2023

Related U.S. Application Data
(60) Provisional application No. 63/444,379, filed on Feb. 9, 2023.

(51) Int. Cl. C08J 11/10; C12P 7/62

(52) U.S. Cl. CPC C08J 11/105; C12P 7/62; B29B 2017/0293; C08J 2367/02

(57) ABSTRACT

A residential plastic recycling unit addresses the challenges of plastic waste management. The unit incorporates a unique enzyme blend capable of degrading plastic containing polyethylene terephthalate into environmentally benign constituents. The unit features precision nozzles for uniform enzyme blend distribution, a grated shelf and receptacle for supporting plastic waste, and a temperature control device with a user interface for precise regulation. After enzymatic degradation, the resulting heterogenous mixture is easily removed and the receptacle cleaned.

### BACKGROUND OF THE INVENTION

The present disclosure relates generally to a residential plastic recycling unit. More particularly, the present disclosure relates to a recycling unit featuring multiple dispensers configured to spray an enzyme capable of breaking down plastic into environmentally friendly components.

A common problem is that people may attempt to recycle plastic items; however, some items may still end up in landfills. Plastics that cannot be recycled may end up in the ocean or different areas of the environment and cause significant damage. People may be upset if they cannot properly recycle all of their plastics.

The inadvertent disposal of non-recyclable plastics into the natural environment has emerged as a significant source of environmental damage. There are a number of potential solutions to such instances of plastic mismanagement. However, the efficacy of any given solution is unpredictable, due at least in part to the complex nature of the technical problem and the difficulty controlling of individual behaviors.

Therefore, what is needed is a residential plastic recycling unit having all of the further described features and advantages.

### SUMMARY OF THE INVENTION

The subject matter of this application may involve, in some cases, interrelated products, alternative solutions to a particular problem, and/or a plurality of different uses of a single system or article.

In one aspect, a recycling unit is disclosed. In this aspect, the recycling unit includes a main body and a plurality of nozzles positioned within the main body. A grated shelf is also located within the main body, and the grated shelf supports a receptacle containing a plastic item. A temperature control device is attached to the main body, and the temperature control device is configured to regulate an internal temperature of the recycling unit.

In another aspect, a method for recycling plastic waste in the recycling unit is disclosed. In this aspect, the method involves a number of steps. The steps include introducing a plastic item into a receptacle within the recycling unit, dispensing an enzyme blend from a plurality nozzles onto the plastic item, and regulating an internal temperature of the recycling unit within a range of 20° C. to 45° C. The method further includes the step of allowing the enzyme blend to degrade the plastic item into a plurality of environmentally benign components.

### BRIEF DESCRIPTION OF DRAWINGS

FIGS. 1, 2, and 3 show examples of the appliance from multiple perspectives, available in different configurations.

FIG. 4 provides a perspective view of one embodiment of the recycling unit.

FIG. 5 provides a cross sectional perspective view of one embodiment of the recycling unit.

FIG. 6 provides a perspective view of one embodiment of the recycling unit interior.

FIG. 7 provides a perspective view of another embodiment of the recycling unit interior.

The foregoing descriptions of specific embodiments of the present invention have been presented for purposes of illustration and description. They are not intended to be exhaustive or to limit the present invention to the precise forms disclosed, and obviously many modifications and variations are possible in light of the above teaching.

The exemplary embodiment was chosen and described in order to best explain the principles of the present invention and its practical application, to thereby enable others skilled in the art to best utilize the present invention and various embodiments with various modifications as are suited to the particular use contemplated.

### DETAILED DESCRIPTION OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some concepts in a simplified form as a prelude to the more detailed description that is presented later.

The detailed description set forth below in connection with the appended drawings is intended as a description of presently preferred embodiments of the invention and does not represent the only forms in which the present disclosure may be constructed and/or utilized. The description sets forth the functions and/or the sequence of steps for constructing and operating the invention in connection with the illustrated embodiments.

Generally, the present disclosure concerns a plastic recycling unit, which provides at least one solution to a known problem. Specifically, the recycling unit provides users with a plastic waste degrading system capable of helping people recycle their plastic items. The recycling unit also features multiple dispensers inside the system that spray a proprietary enzyme on the plastics to help break it down into environmentally friendly components. The recycling unit or system also functions like an incubator for plastics, resembling a miniature or refrigerator other similar appliance that can be used in homes, offices, apartments, and more. In short, the system prevents plastics from ending up in landfills, improving environmental consciousness.

The residential plastic recycling unit is a plastic waste degrading device comprised of a body with a reservoir that receives an enzyme blend. The blend is sprayed onto plastic waste placed within the system receptacle, wherein the runoff is collected in a catch pan with drainage holes. The runoff can be transferred to another location via a drainage hose. The device is also comprised of an automated-temperature regulation feature.

In certain embodiments, the enzyme blend contained within the reservoir of the body may contain one or more enzymes derived from a bacterium known as Ideonella sakaiensis 201-F6. This bacterium possesses the capacity to produce enzymes that are critical in the degradation of polyethylene terephthalate (“PET”), a ubiquitous polymer utilized extensively in plastic products worldwide. The accumulation of PET waste poses a significant environmental concern.

Specifically, Ideonella sakaiensis 201-F6 produces two enzymes, which may be further synthesized and/or purified before being used in the enzyme blend. The two enzymes are capable of breaking down PET into its two environmentally benign monomers: terephthalic acid (“TPA”) and ethylene glycol (“EG”), both of which are originally sourced from raw petroleum.

The primary enzyme produced by the bacterium, denoted as PET hydrolase (“PETase”), assumes the pivotal role of initiating the degradation of PET. After an incubation period of approximately eighteen (18) hours at a neutral pH, PETase transforms PET into a primary reaction intermediate known as mono (2-hydroxyethyl) terephthalic acid (“MHET”). This enzymatic reaction may be most efficient within a temperature range spanning from 20° C. to 45° C. (approximately 68° F. to 113° F.), inclusive. This temperature range may align with the optimal flexibility of PET's polyester chains, facilitating the degradation process.

Once PET has been successfully degraded to MHET by the action of PETase, a secondary enzyme e produced by Ideonella sakaiensis 201-F6, termed MHET hydrolase (“MHETase”), becomes critical to further decomposition. MHETase catalyzes the further hydrolyzation of MHET, ultimately yielding the environmentally benign monomers, TPA and EG.

While the presently preferred embodiments highlight the utilization of PETases and MHETases produced by Ideonella sakaiensis 201-F6 as well as specific reaction conditions and reaction products, it should be expressly understood that these examples are not intended to restrict the scope of the present disclosure. The composition of the enzyme blend within the recycling unit need not be confined solely to PETases and MHETases, especially to those PETases and MHETases produced by Ideonella sakaiensis 201-F6. Similarly, the reaction conditions, including time and temperature ranges, and the reaction products, including TPA and EG, are not confined exclusively to the parameters and chemicals described herein. The versatility of the recycling unit enables the incorporation of alternative enzymes and the customization of reaction conditions and resulting products, as may be deemed suitable for the degradation or breakdown of plastic in various contexts and applications.

The appliance acts as an incubator for the biological breakdown of plastic after the application of the enzymatic solution that coats the plastic. The appliance will have multiple shelves for evenly spaced plastic containers. The inside will have evenly distributed nozzles from which the solution will be sprayed. There may be a UV light at the top to initiate the reaction. Under the shelves will be similar receptacles for the collection of biological material after the plastic has degraded into a form that can be discarded properly into the environment. The unit will be temperature controlled, possibly with a multiple temperature scheduled setting.

In one embodiment, the recycling unit may incorporate a temperature control device configured to provide precise regulation of the appliance's internal temperature. In one embodiment, this temperature control device may critically ensure that the unit operates within the temperature range of 20° C. to 45° C., thus optimizing the enzymatic decomposition of PET by PETase.

The temperature control device may be equipped with a user interface and a plurality of controls that allow the unit's temperature settings to be configured and customized. For example, through the interface and controls, users may set and monitor the internal temperature, establish temperature schedules, and specify the duration for which the desired temperature should be maintained.

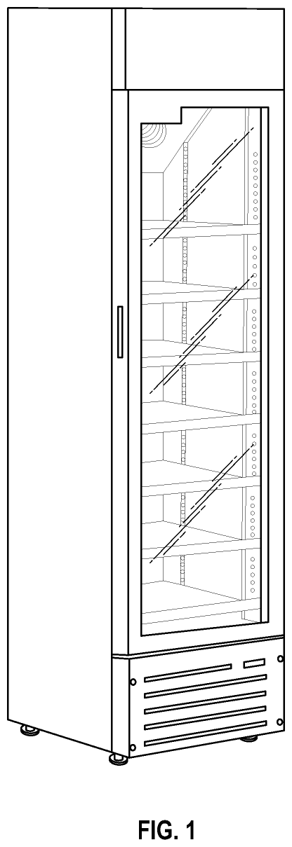

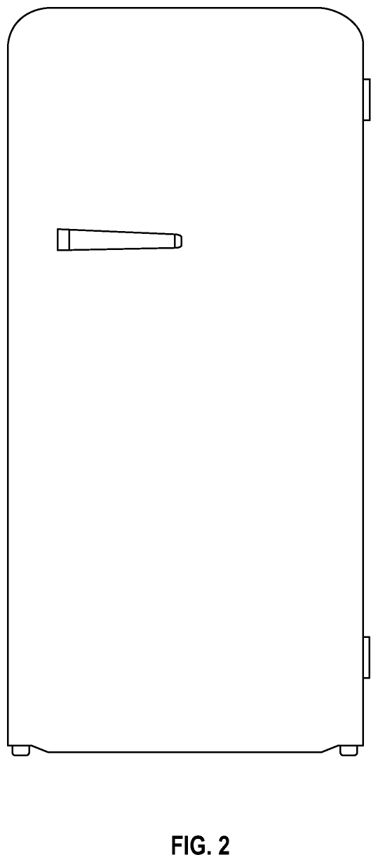

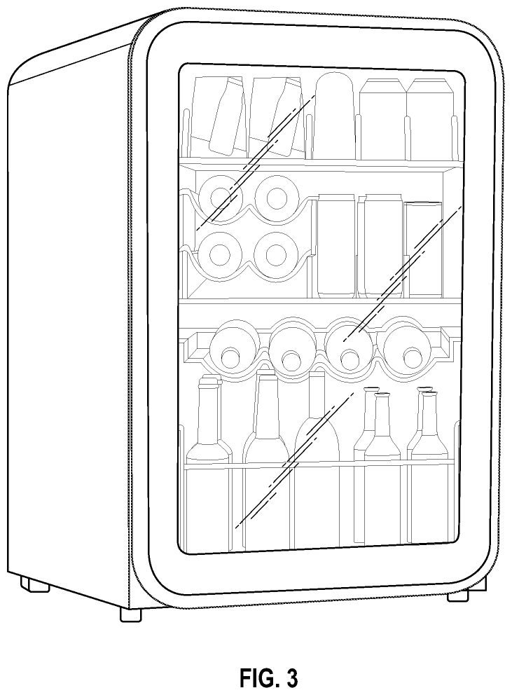

Exact size, measurement, construction, and design specifications may vary upon further development and manufacturing. For example, to facilitate a more comprehensive understanding of the residential plastic recycling unit and to emphasize the adaptable nature of its components, FIGS. 1-3 depict various perspective views of different embodiments of the recycling unit. These illustrations serve the purpose of demonstrating that the unit's physical appearance and specific component arrangements may vary while remaining within the scope of the present disclosure.

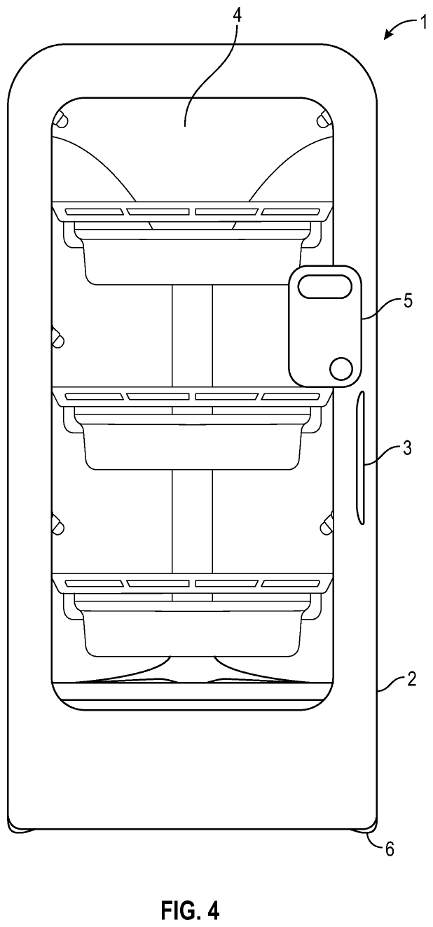

FIG. 4 offers a detailed perspective view of a presently preferred embodiment of the recycling unit 1, providing a closer look at its structural attributes and functional components. In this embodiment, the recycling unit 1 is composed of two primary elements: the main body 2 and an accompanying door 3 that is securely affixed to the main body 2. Notably, the door 3 features a prominent transparent panel 4 designed to allow users to clearly observe the contents within the main body 2. The transparent panel 4 can be manufactured from a range of suitable materials, including, but not limited to any type of glass, acrylic, polycarbonate, and the like, ensuring both durability and visual clarity. However, in at least one embodiment, it is critical that the interior face of the transparent panel 4 not be manufactured from plastic or any material containing PET to prevent inadvertent degradation of the panel 4.

Integrated into the door 3 is a temperature control device 5, which plays a key role in automating and maintaining a controlled environment within the main body 2. This temperature control device 5 empowers the recycling unit 1 to precisely regulate its internal temperature, optimizing the conditions required for the efficient biological breakdown of the plastic materials. In an alternative embodiment, the temperature control device 5 may be attached to the main body 2 instead of the door 3.

As a further part of the structural design of the unit 1, a plurality of stands 6 are incorporated into and provide essential support for the main body 2. These stands 6 ensure the stability and secure placement of the recycling unit 1 within its intended environment, be it a household, office, or other settings.

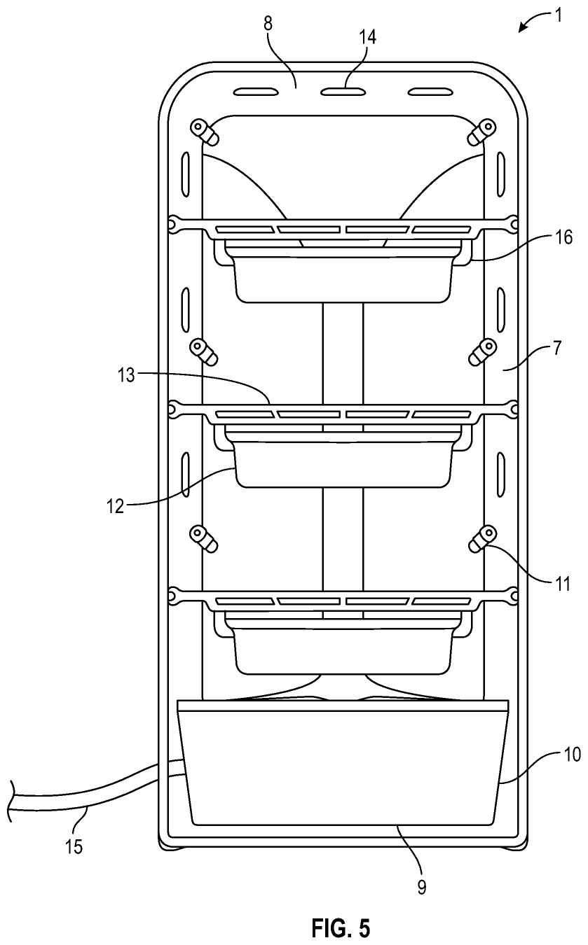

FIG. 5 offers a cross-sectional perspective of the recycling unit 1, allowing for a comprehensive view of its interior features. Within the unit 1, several structural elements define its configuration. The primary structural components encompassing the interior space of the unit 1 include at least three side walls 7, a top wall 8, and a bottom wall 9. These walls, in conjunction with the integral door 3 shown in FIG. 4, collectively demarcate and enclose the functional workspace of the recycling unit 1.

Situated on the lowermost portion of the unit 1, proximal to the bottom wall 9, a dedicated catch pan or compartment 10 serves the key role of capturing liquid runoff. The runoff results from the distribution of a spray solution from a multitude of strategically positioned nozzles 11. Notably, these nozzles 11 are securely affixed to at least two of the side walls 7 and are evenly arranged throughout the internal space of the unit 1. This distribution ensures a uniform application of the spray solution, promoting efficient plastic degradation.

The spray solution is specifically engineered to facilitate degradation of plastic materials into environmentally benign components. This solution is dispensed through the array of nozzles 11 strategically positioned throughout the interior of the unit 1. Crucially, the interior of the unit 1 incorporates receptacles 12 supported by grated shelves 13. The spray solution passes through the openings in the grated shelves 13, thus efficiently filling the receptacle 12.

Similarly, the presence of UV lights 14 further enhances the plastic degradation process. The UV lights 14 may be used to initiate and accelerate the breakdown of plastic materials, contributing to the overall efficiency of the unit 1. Also, in this embodiment, to manage runoff generated during the degradation process, the recycling unit 1 incorporates a drainage system. The drainage system includes a drainage hose 15 securely attached to an aperture defined by the compartment 10. This drainage hose 15 facilitates the transfer of liquid runoff from the compartment 10 to the external environment through an aperture defined in the main body 2, ensuring continuous operation of the recycling unit 1.

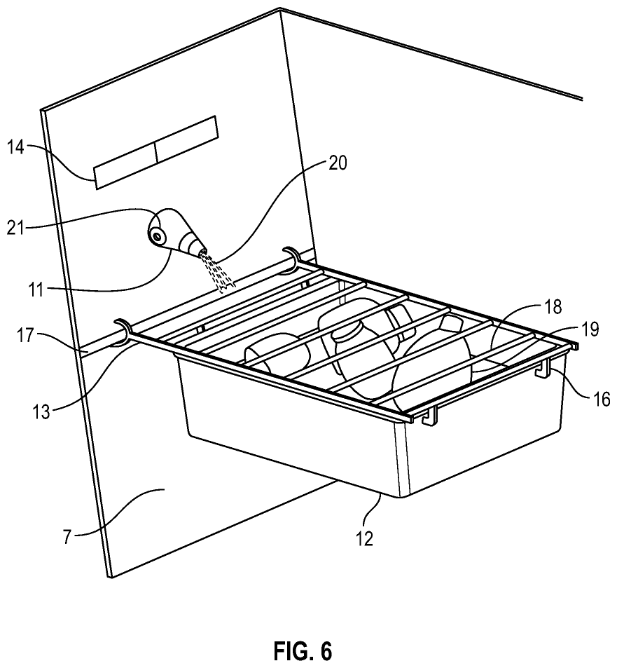

FIG. 6 offers another perspective view of an embodiment of the interior of the recycling unit 1, further illustrating its internal design and component interactions. In this embodiment, the shelf 13 is affixed to the sidewall 7 of the unit 1. The shelf 13 serves as a platform supporting a receptacle 12, which is securely held in place by a series of hangers 16. In this embodiment, two pairs of hangers 16 are situated proximal to opposing ends of the shelf 13. Furthermore, protrusions 17 integrated into at least two of the sidewalls 7 stabilize and secure the shelf 13 in its designated position. The shelf 13 features grating 18, revealing the plastic waste or items 19 contained within the receptacle 12.

The receptacle 12 may first be detached from the shelf 13 before the plastic items 19 are placed therein. This may be achieved by sliding the receptacle 12 out along the hangers 16, creating an accessible space for the placement of one or more plastic items 19. Once the plastic items 19 are placed within the receptacle 12, it can be slid back into position, with the hangers 16 providing support. When the receptacle 12 is securely positioned beneath the grating 18, the plastic degradation process may be initiated by either manual inputs to or automatic outputs from the temperature control device 5 for the unit 1.

Once the process is initiated, the nozzles 11 are configured to dispense a solution 20 enriched with a specialized enzyme blend designed to degrade plastic. In this preferred embodiment, the enzymes within the spray solution 20 are PETase and MHETase. A UV light 14 positioned above the nozzle 11 on the sidewall 7 plays a dual role of illuminating and accelerating the degradation process of the plastic items 19. Furthermore, the nozzle 11 can be adjusted vertically by rotation about a pivot point 21. This configuration enables fine-tuning of the angle at which the solution 20 is dispensed, ensuring optimized interaction between the solution 20 and the plastic items 19 through the grating 18.

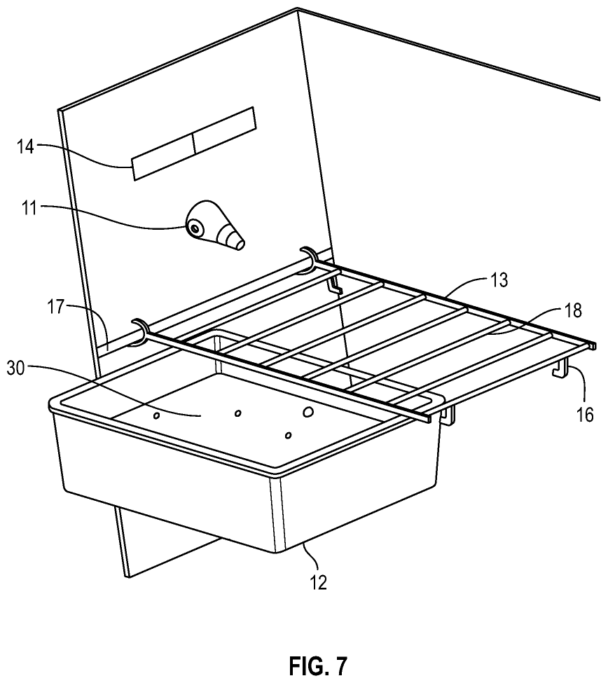

FIG. 7 provides a close-up perspective view of the receptacle 12 following the completion of the plastic degradation process by the spray solution 20. In this embodiment, a critical feature is the coordinated operation of the nozzles 11 and the UV lights 14, configured to pause the dispensing of solution 20 and cease emitting light.

As a result of the coordinated operation of the nozzles 11, UV lights 14, and the solution 20, which may be accomplished through manual inputs to or automatic outputs from the temperature control device, the once distinct plastic items 19 are transformed into a new heterogenous mixture 30. This mixture 30 may be naturally decomposable, as it may primarily contain TPA, EG, and water, all of which are environmentally benign.

To facilitate the removal of this environmentally friendly mixture 30 from the receptacle 12, a straightforward procedure is followed. First, the receptacle 12 is detached from the shelf 13, a step which can be accomplished by sliding it out along the hangers 16. Once the mixture 30 is extracted, the receptacle 12 itself may undergo a thorough cleaning, ensuring its preparation for subsequent use. With the receptacle 12 cleaned, it can be easily reattached to the shelf 13, configured to repeat the aforementioned recycling process.

In this embodiment, it is critical that the shelf 13 and the other components located within the interior of the recycling unit 1, including, but not limited to, the internal walls of the unit 1, the interior face of the transparent panel 4, the receptacles 12, and the compartment 10 are not constructed from plastic or any material containing PET. Consequently, the presence of PETase in the solution 20 does not pose any risk of degradation to the shelf 13, its accompanying grating 18, or any other internal components of the unit 1. This resilience of the material within the recycling unit 1 facilitates extended use of the appliance.

Nevertheless, to ensure the optimal performance of the recycling process, the shelf 13 and other components within the recycling unit 1 may require periodic cleaning. For example, the shelf 13 and other interior components of the unit 1 may be cleaned within the recycling unit 1 itself. Alternatively, the shelves 13 and receptacles 12 may be detached from the unit 1 to facilitate a more thorough cleaning. Removal of the shelf 13 specifically can be achieved by smoothly sliding it out along the protrusions 17 on the sidewalls 7, ensuring straightforward access for maintenance.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

While several variations of the present disclosure have been illustrated by way of example in preferred or particular embodiments, it is apparent that further embodiments could be developed within the spirit and scope of the present disclosure, or the inventive concept thereof. However, it is to be expressly understood that such modifications and adaptations are within the spirit and scope of the present disclosure, and are inclusive, but not limited to the following appended claims as set forth.

What is claimed is:

1. A recycling unit, comprising:
a main body and a plurality of nozzles positioned within the main body;
a grated shelf within the main body, the grated shelf supporting a receptacle containing a plastic item; and
a temperature control device attached to the main body;
wherein the temperature control device is configured to regulate an internal temperature of the recycling unit.

2. The recycling unit of claim 1 further comprising a reservoir integrated within the main body, the reservoir storing a spray solution.

3. The recycling unit of claim 2 wherein the spray solution comprises an enzyme blend capable of degrading the plastic item.

4. The recycling unit of claim 1 wherein the plurality of nozzles are configured to dispense a spray solution onto the plastic item.

5. The recycling unit of claim 4 wherein the spray solution comprises at least one of a PETase and a MHETase.

6. The recycling unit of claim 1 further comprising a ultraviolet light positioned above the plurality of nozzles.

7. The recycling unit of claim 1 wherein the receptacle and the grated shelf are configured to be removed from the main body.

8. The recycling unit of claim 1 wherein the receptacle and grated shelf do not comprise plastic.

9. The recycling unit of claim 1 further comprising a door attached to the main body.

10. The recycling unit of claim 9 wherein the door comprises a transparent panel.

11. The recycling unit of claim 1 wherein the main body comprises a plurality of sidewalls, a top wall, and a bottom wall.

12. The recycling unit of claim 11 further comprising a catch pan proximal to the bottom wall.

13. The recycling unit of claim 11 wherein at least two of the plurality of sidewalls define a protrusion for supporting the grated shelf.

14. The recycling unit of claim 1 further comprising a drainage hose for transferring a liquid runoff from the recycling unit.

15. The recycling unit of claim 1 wherein the grated shelf comprises a plurality of hangers supporting the receptacle.

16. A method for recycling plastic waste in a recycling unit, comprising:
introducing a plastic item into a receptacle within the recycling unit;
dispensing an enzyme blend from a plurality of nozzles onto the plastic item;
regulating an internal temperature of the recycling unit; and
allowing the enzyme blend to degrade the plastic item into a plurality of environmentally benign components.

17. The method of claim 16 wherein the plastic item is introduced into the receptacle after the receptacle is detached from a grated shelf supporting the receptacle.

18. The method of claim 16 wherein the degradation of the plastic item occurs over a period of time.

19. The method of claim 18 wherein the internal temperature is maintained at a constant temperature for the period of time.

20. The method of claim 16 further comprising adjusting at least one of the plurality of nozzles.
